(19) United States
(12) Patent Application Publication
(10) Pub. No.: US 2014/0023676 A1
(43) Pub. Date: Jan. 23, 2014

(54) COSMETIC COMPOSITION CONTAINING SPIRULINA, AND COSMETIC TREATMENT METHOD

(71) Applicants: Dolivar Coraucci Neto, Sertaozinho (BR); Flávio Bueno de Camargo, JR., Piracicaba (BR); Patricia Berardo Gonçalves Maia Campos, Ribeirao Preto (BR)

(72) Inventors: Dolivar Coraucci Neto, Sertaozinho (BR); Flávio Bueno de Camargo, JR., Piracicaba (BR); Patricia Berardo Gonçalves Maia Campos, Ribeirao Preto (BR)

(73) Assignee: Ouro Fino Participacoes e Empreedimentos S/A, Ribeirao Preto (BR)

(21) Appl. No.: 14/009,710

(22) PCT Filed: Apr. 3, 2012
(86) PCT No.: PCT/BR12/00095

§ 371 Date: Oct. 3, 2013

(30) Foreign Application Priority Data
Apr. 4, 2011 (BR) PI 1101659-0

(51) Int. Cl. A61K 8/97; A61Q 19/08

(52) U.S. Cl. CPC A61K 8/975; A61Q 19/08
USPC 424/195.17

(57) ABSTRACT

The present invention relates to a cosmetic composition for topical application containing Spirulina, which has as main objective to combat the action of free radicals that act on aging, in addition to providing hydration, protection and improvement of the general conditions of the skin. The composition comprises Spirulina as a dry extract in concentrations ranging from 0.1 to 5.0% by weight, and cosmetically acceptable vehicles.

### FIELD OF THE INVENTION

The present invention relates to a cosmetic composition for topical application containing Spirulina, which has as main objective to combat the action of free radicals that act on aging, in addition to providing hydration, protection and improvement of the general conditions of the skin.

### BACKGROUND OF THE INVENTION

The skin is a complex organ that is comprised of a metabolically active barrier. Its main function is to protect the internal organs against ultraviolet light, mechanical and chemical attacks and excessive dehydration against microorganisms. Therefore, the maintenance of the structural integrity of the skin as well as the restoration of its barrier properties becomes extremely important for a healthy skin.

In this context, cosmetology is gaining more and more prominence once the skin reflects our health and our quality of life. Thus, a healthy lifestyle combined with a balanced diet and the use of products for skin protection against environmental aggressions and which can promote different benefits to the skin has been widely reported worldwide.

Every year there are new cosmetic products containing different active substances, being the vitamins such as A, B and C, and those of the B complex the ones which play a major role by presenting moisturizing and emollient effects, antioxidant and protective activity against the damage caused by the ultraviolet rays and keratinization and melanogenesis control. However, besides vitamins, other active substances such as amino acids, proteins and plant extracts have been used to improve the overall appearance of the skin as it can act in moisturizing and protecting the skin from environmental aggression, maintaining its eudermia.

Furthermore, it should be noted that extracts rich in proteins are considered potential raw materials for topical use according to the benefits they can provide to the skin tissue, both in terms of prevention and for the maintenance and treatment of the skin, keeping it healthy and in good condition.

The current trend in terms of formulation is to allow several active substances to be conveyed in the same product, targeting the synergism to obtain a product with multifunctional characteristics, such as moisturizing action, protection of the barrier function of the skin, and may also act in cell renewal.

Another strong trend in cosmetic formulations is related to the use of active substances of natural origin, which are being widely applied in the industry and have a great acceptance by the consumer market. There are thousands of examples of these actives for use in cosmetic products. Vegetable oils are highly resistant to oxidation, plant extracts that maintain full integrity of its active components, various species of algae and also the alpha-hydroxy acids such as glycolic acid and fruit acids extracted from sugar cane and fruits, which have revolutionized the cosmetic industry by the amount of scientific papers proving its effectiveness in treating the skin and certain dermatoses such as, for example, ichthyosis.

Among the actives of natural origin, Spirulina (or Arthrospira), which belongs to the group of Cyanobacterium, also known as Cyanophyta or as a group of blue-green algae, has been widely used as a human dietary supplement, mainly as pills or tablets of pressed Spirulina, as well as animal food supplement.

The use of Spirulina as a food supplement is due to its nutritional characteristics. Spirulina contains between 50 and 70% of its dry weight in protein, amount which in general is higher than that of other protein sources. Among the amino acids found in Spirulina we can highlight methionine, glycine, lysine and gamma-linolenic acid (GLA). In addition to the high concentration of proteins, it has between 8 and 14% of polysaccharides, of which the main monomers are glucose, galactose, mannose and ribose, and about 6% of lipids, but both their quantities and their compositions vary depending on the conditions of cultivation, mainly light and nitrogen. If light is scarce, the content of lipids as an energy reserve will be increased. Due to being a photoautotroph organism, Spirulina has high concentrations of pigments, including β-carotene, i.e., pro-vitamin A. In addition, Spirulina has B vitamins in its composition, and the non-animal body is the one with a greater content of vitamin B12 or cobalamine.

Since Spirulina has a composition rich in pro-vitamin A, vitamins of the B complex, proteins and polysaccharides such as glucose, galactose, mannose and ribose, when used as an active in cosmetic formulations it can provide the skin with moisturizing and emollient effect, antioxidant activity, and protective activity.

Another prominent characteristic in the use of Spirulina in cosmetics is the possibility of developing formulations with multifunctional, stable, safe characteristics, and at a lower cost, since the addition of active substances in cosmetic formulations may cause instability to the same, like the decrease in viscosity and changes in their rheological characteristics in general. Thus, the greater the number of active substances present in the formulation, the greater the chances of the formulation to present stability problems.

In the past, natural products were used empirically, but as the consumers looked for natural products of higher purity, safety and effectiveness, there was a need to increase scientific and technological efforts to evaluate and control the quality of the products containing natural substances. Example of these uses of the past can be found in the documents PI0605365-3 and 3-PI0705238 which deal with handicraft production methods and the application of Spirulina “in natura” to cosmetic creams. These documents show that one of the major technical problems to be solved is related to the production of a dry extract of Spirulina without causing cell death and the consequent loss of its nutrients, notably the polysaccharides which can be degraded. The solution sought, therefore, was to try to formulate Spirulina in its living form, i.e., “in natura”, together with other cosmetic actives. However, it is apparent that such a solution is handicraft and has no how to be applied to the industrial production of a cosmetic formulation due to the loss of stability since it needs to be stored in controlled temperature at about 8° C. to achieve a shelf life greater than 30 days.

At first, it may seem contradictory to associate the words natural and technology, but the effectiveness of the products that contain natural ingredients depends on both the purity of the raw materials and the development of a suitable formulation. Moreover, as mentioned, the addition of active substances to cosmetic formulations generally causes instability to the same, noting that the higher the number of active substances present in the formulation, the greater the chances of the formulation to present stability problems.

That is why the development of new cosmetic products must take into consideration, among others, the type of formulation, the manufacturing process, the purpose of use, the skin type and the compatibility among the possible active substances to be added to them, which leads to the need for stability studies and evaluation of effectiveness.

Thus, a major challenge for researchers in the cosmetic field is to develop formulations that offer various benefits to the skin using a smaller number of active substances in its composition and, in this context, once Spirulina is a substance of natural origin that has in its composition many substances that can bring benefits to skin, it appears as a compound of great potential for the development of such formulations.

### SUMMARY OF THE INVENTION

Considering the information disclosed above, it has now become a reality, through preclinical studies of antioxidant activity and clinical effectiveness, obtaining industrial cosmetic formulations based on Spirulina presenting stability and effectiveness in moisturizing, protecting, and improving the microrelief and the general conditions of the skin. These new stable topical formulations containing Spirulina are innovative and of great importance, once effective products with multifunctional features were obtained.

So, a cosmetic composition has just been developed, comprising dry extract of Spirulina, which has a good concentration of nutrients that can really bring benefits to skin when conveyed in cosmetic formulations, without the need to keep the algae alive and increasing the shelf live of the formulation without having to store it under refrigeration.

The cosmetic composition object of the present invention has proved, by means of studies of assess of the effectiveness to be described here, that it maintains the nutrients at appropriate concentrations for use in cosmetic products.

The present invention also contemplates a method of cosmetic treatment comprising applying the composition according to the present invention.

### DETAILED DESCRIPTION OF THE INVENTION

In accordance with the above objectives, the cosmetic composition according to the present invention comprises Spirulina as a dry extract in a concentration varying from 0.1 to 5.0% by weight and cosmetically acceptable vehicles with sensory suited to their purposes of use.

The other components used in the formulation of the present invention include Paramul (autoemulsifying nonionic wax) Aristoflex (acrylate polymer), propylene glycol, glycerin, phenoxyethanol and parabens, Net FS (silicone microemulsion), DC 9040 (Silicon), DC 245 (volatile silicon), DC 9011 (silicon) DC 200/50 (silicon) Dragoxat (octyl octanoate) and emulgin 40OE (hydrogenated and ethoxylated castor oil).

According to the present invention, the products listed below have the following meanings according to INCI—International Nomenclature of Cosmetic Ingredient:

DC 9040—Cyclopentasiloxane

DC 9011—Cyclopentasiloxane (and) PEG-12 Dimethicone Crosspolymer

DC 245—Cyclomethicone

DC 200/50—Dimethicone

Aristoflex—Ammonium Acryloyldimethyltaurate/VP Copolymer

Paramul—Cetearyl Alcohol (and) Ceteareth-20

Preferably, said components are present in the cosmetic composition of the present invention in the following ratios by weight: Paramul (0 to 12.0%), Aristoflex (0-2.0%), propylene glycol (4.0-6.0%), Glycerin (6.0%), Phenoxyethanol and parabens (0.8%) Net FS (4.0-5.0%), DC 9040 (10.0 to 21.0%), DC 245 (5.0-7.0%), DC 9011 (0-5.0%) DC 200/50 (0 to 3.0%), Dragoxat (3.0%) and emulgin 40OE (0 to 3.0%).

In a preferred form, the cosmetic composition of the present invention is formulated as an emulsion comprising, in percentage by weight, from 0.1 to 5.0% of Spirulina as a dry extract, 12% of Paramul, 4.0% of Propylene Glycol, 6.0% of Glycerin, 0.8% of phenoxyethanol and parabens, 5.0% of Net FS, 10.0% of DC 9040, 5.0% of DC 245, 5.0% of DC 9011, 3.0% of DC 200/50, 3.0% of Dragoxat and distilled and deionized water (qs 100).

Giving emphasis to the aforementioned preference, the cosmetic composition according to the present invention is formulated as a cream-gel comprising, in percent by weight,

from 0.1 to 5.0% of Spirulina as a dry extract, 2.0% of Aristoflex, 6.0% of Propylene Glycol, 6.0% of Glycerin, 0.8% of phenoxyethanol and parabens, 4.0% of Net FS, 21.0% of DC 9040, 7.0% of DC 245, 3.0% of Dragoxat, 3.0% of Emulgin 40OE and distilled and deionized water (qs 100).

The cosmetic composition containing Spirulina according to the present invention has multifunctional characteristics, is very stable and safe, and can be produced at low cost. Moreover, being a product of natural origin, the process for the production of Spirulina and its dry extract will not cause negative impacts on nature, which is a concern of environmentalists and protectors of the environment when they question the impacts of commercial extraction by the industries on the rivers and forests.

Therefore, in order to prove stability, antioxidant potential, skin compatibility and effectiveness in the short and long term, as the purposes of the present invention, various studies and tests have been developed with various cosmetic compositions containing Spirulina, as a dry extract, so that it was possible to reach those which reveal a greater expression of such qualities.

The studies and tests for these purposes will be illustrated below as non-limiting examples of the scope of protection conferred by the claims attached, but showing the results and effectiveness of the cosmetic composition according to the present invention. All percentages are by weight.

### Example 1

### Evaluation of the Stability of Spirulina in Cosmetic Formulations

The following formulations were prepared (% w/w) as shown below in Table 1:

TABLE 1

| | Formulation | | |
|---|---|---|---|
| | F1 | F2 | F3 |
| Spirulina solubilized in | — | 0.1 | 0.1 |
| water, 1:10 | | | |
| DC 9040 | 65 | qs 100 | 25 |
| DC 245 | 15 | — | 37.1 |
| DC 9011, 10% | 10 | — | — |
| Glycerin | 2.5 | 2.5 | 2.5 |
| Propylene glycol | 2.5 | 2.5 | 2.5 |
| Phenova | 0.8 | 0.8 | 0.8 |
| (phenoxyethanol and | | | |
| parabens) | | | |
| Uniox A | — | — | 12 |
| (autoemulsifying | | | |
| nonionic wax) | | | |
| Water | — | — | 20 |

Comments:

In formulation F1 it was not possible to incorporate Spirulina. Formulation F2 showed no change in color after 3 days of storage at room temperature and at 45° C., but had phase separation, which is undesirable. Formulation F3 showed no change in color after 5 days of storage at room temperature and at 45° C., but had phase separation, which is undesirable.

Subsequently, another 4 formulations were prepared (% w/w), which are described below in Table 2:

TABLE 2

| | Formulation | | | |
|---|---|---|---|---|
| | F4 | F5 | F6 | F7 |
| Spirulina | 0.1 | 0.1 | 0.1 | 0.1 |
| Paramul | 12 | 12 | 10 | — |
| (autoemulsifying wax) | | | | |
| Aristoflex | — | — | — | 2.0 |
| (acrylate polymer) | | | | |
| Propylene glycol | 3.0 | 4.0 | 4.0 | 6.0 |
| Glycerin | 3.0 | 6.0 | 6.0 | 6.0 |
| Phenoxyethanol and Parabens | 0.8 | 0.8 | 0.8 | 0.8 |
| Net FS | 5.0 | 5.0 | 5.0 | — |
| (silicon microemulsion) | | | | |
| DC 9040 | 10 | 10 | 15 | 21 |
| DC 245 | 10 | 5 | 5 | 7 |
| DC 9011 | 5 | 5 | 5 | — |
| DC 200/50 | — | 3 | 3 | — |
| Dragoxat | — | 3 | 3 | 3.0 or |
| (octyl octanoate) | | | | lanol |
| Emulgin 40OE | — | — | — | 3.0 |
| (hydrogenated and ethoxylated | | | | |
| castor oil) | | | | |
| Distilled and deionized water | qs 100 | qs 100 | qs 100 | qs 100 |

Formulation F4 was not stable in the centrifugation test, as it showed phase separation, and formulations F5, F6 and F7 were stable, showing no phase separation. Thus, the formulations F5, F6 and F7 were tested for stability by visual evaluation, the samples being stored at room temperature in greenhouses at 37° C. and 45° C.

In these tests, such formulations showed color change in time 4 days, and formulations F5 and F7 showed a lighter tone. This lighter colored continued until time 15 days at all temperatures. After 21 days of storage, the formulations that were kept at 45° C. showed a very small clearance in relation to those kept at 37° C. After 41 days the formulations showed no color changes. Formulation F6, when stored at 45° C., showed a slight surface dryness and a little change in consistency.

Thus, formulations F5 and F7 were considered more stable. These formulations were prepared again and subjected to stability tests without the addition of Spirulina to serve as a control. After 10 days of storage the formulations showed no color changes. After 91 days of storage, the formulations showed no color change, only formulation F5 stored at 45° C. showed a slight dryness on the surface.

Formulations F5 (pH 6.4) and F7 (pH 6.0) were then selected for testing safety by determining the dermal compatibility.

### Example 2

### Evaluation of the Antioxidant Potential In Vitro of Spirulina Samples

The test was performed with the equipment Autolumat LB953 Luminometer EG&G Berthold. Free radicals were produced with the hydrogen peroxide and the enzyme peroxidase. Luminol was used as a probe which reacted with the free radical which produces a photon that is captured by the equipment. If the substance tested has antioxidant action, it reacts with the free radical and therefore less free radicals will react with luminol, decreasing the number of photons emitted. All tests were performed in triplicate.

2.1. Evaluation Results of the Antioxidant Potential In Vitro of the First Spirulina Sample Received (AGA):

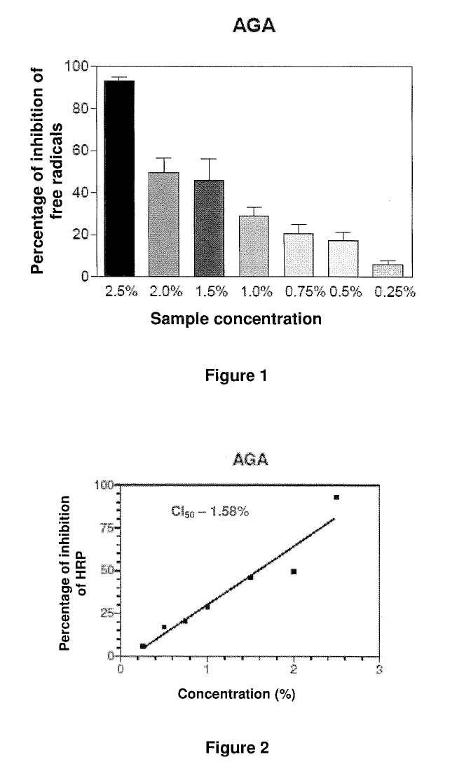

The graph in FIG. 1 shows the different percentages of Spirulina used in the experiment, as shown in Table 3.

TABLE 3

| Concentration | Actual concentration after | % Inhibition of free |
|---|---|---|
| (Spirulina) | dilution (Spirulina) | radicals |
| 2.5 | 0.025 | 93.2 |
| 2.0 | 0.020 | 49.7 |
| 1.5 | 0.015 | 46 |
| 1.0 | 0.010 | 29.2 |
| 0.75 | 0.0075 | 20.6 |
| 0.50 | 0.0050 | 17.2 |
| 0.25 | 0.0025 | 6 |

By means of the values obtained, we calculated the percentage of Spirulina required to inhibit 50% of free radicals. The value obtained was 1.58%, i.e., under the experimental conditions to inhibit 50% of the free radicals a concentration of 1.58% of Spirulina was required (actual concentration after dilution 0.0158%). The value curve is illustrated in FIG. 2.

2.2. Evaluation Results of the Antioxidant Potential In Vitro of the Second Spirulina Sample (Higher Number of Washes) (AGN):

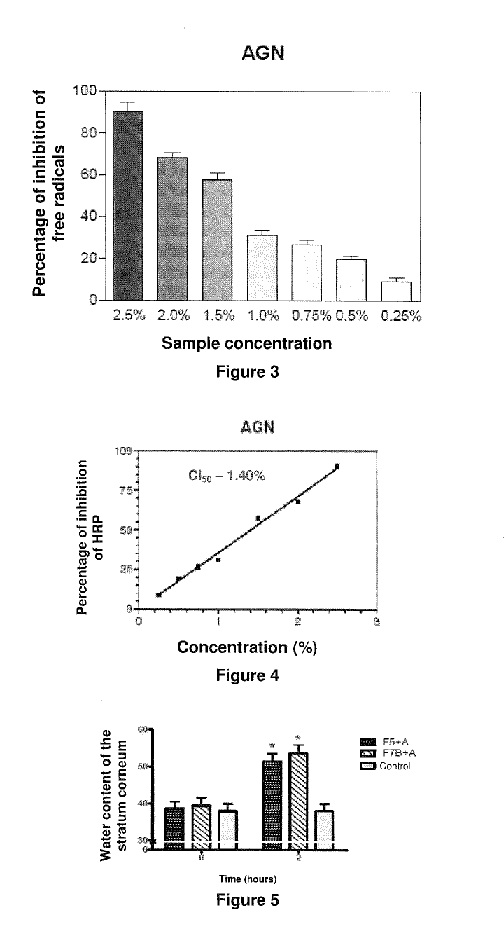

The graph in FIG. 3 shows the different percentages of Spirulina used in the experiment, as shown in Table 4.

TABLE 4

| Concentration | Actual concentration after | % Inhibition of free |
|---|---|---|
| (Spirulina) | dilution (Spirulina) | radicals |
| 2.5 | 0.025 | 90.3 |
| 2.0 | 0.020 | 68.3 |
| 1.5 | 0.015 | 57.5 |
| 1.0 | 0.010 | 31.1 |
| 0.75 | 0.0075 | 26.88 |
| 0.50 | 0.0050 | 19.80 |
| 0.25 | 0.0025 | 9.23 |

By means of the values obtained, we calculated the percentage of Spirulina required to inhibit 50% of free radicals. The value obtained was 1.40%, i.e., under the experimental conditions to inhibit 50% of the free radicals a concentration of 1.40% of Spirulina was required (actual concentration after dilution 0.014-%). The value curve is illustrated in FIG. 4.

### Example 3

### Evaluation of Skin Compatibility of Cosmetic Formulations with the Addition of 0.1% of Spirulina

TABLE 5

Formulations

| | F5 | F7 |
|---|---|---|
| Spirulina | 0.1 | 0.1 |
| Paramul | 12 | — |
| (Autoemulsifying nonionic wax) | | |
| Aristoflex | — | 2.0 |
| (acrylate polymer) | | |
| Propylene glycol | 4.0 | 6.0 |
| Glycerin | 6.0 | 6.0 |
| Phenoxyethanol and Parabens | 0.8 | 0.8 |
| Net FS | 5.0 | — |
| (silicon microemulsion) | | |
| DC 9040 | 10 | 21 |
| DC 245 | 5 | 7 |
| DC 9011 | 5 | — |
| DC 200/50 | 3 | — |
| Dragoxat | 3 | 3.0 |
| (octyl octanoate) | | |
| Emulgin 40OE | — | 3.0 |
| (hydrogenated and ethoxylated | | |
| castor oil) | | |
| Distilled and deionized water | qs 100 | qs 100 |

In the evaluation tests of skin compatibility, formulations F5 (emulsion) and F7 (gel-cream) were considered safe for use on human skin.

### Example 4

### Sensory Evaluation of Cosmetic Formulations with or without the Addition of 0.1% of Spirulina

For sensory evaluation we prepared 2 more formulations F5B and F7B. Thus, we evaluated the sensory characteristics of emulsions and F5 and F5B and cream-gels F7 and F7B.

TABLE 7

| | Formulations | | | |
|---|---|---|---|---|
| | F5 | F5B | F7 | F7B |
| Spirulina | 0.1 | 0.1 | 0.1 | 0.1 |
| Paramul | 12 | 12 | — | — |
| (autoemulsifying wax) | | | | |
| Aristoflex | — | — | 2.0 | 2.0 |
| (polymer) | | | | |
| Propylene glycol | 4.0 | 6.0 | 6.0 | 6.0 |
| Glycerin | 6.0 | 6.0 | 6.0 | 6.0 |
| Phenoxyethanol and Parabens | 0.8 | 0.8 | 0.8 | 0.8 |
| Net FS | 5.0 | 7.0 | — | 4.0 |
| (silicon microemulsion) | | | | |
| DC 9040 | 10 | 10 | 21 | 21 |
| DC 245 | 5 | 7 | 7 | 7 |
| DC 9011 | 5 | 5 | — | — |
| DC 200/50 | 3 | 3 | — | — |
| Dragoxat | 3 | 6 | 3.0 | 3.0 |
| (octyl octanoate) | | | | |
| Emulgin 40OE | — | — | 3.0 | 3.0 |
| (hydrogenated and ethoxylated | | | | |
| castor oil) | | | | |
| Distilled and deionized water | qs 100 | qs 100 | qs 100 | qs 100 |

In this study, a standardized amount (200 mg) of the formulations described above was applied in different regions in the lower middle part of the forearms and then the volunteers received a Sensory Evaluation Form according to the model below, where they answered questions assigning grades.

TABLE 8

| | | | | |
|---|---|---|---|---|
| Characteristics | F5 | F5B | F7 | F7B |

Feel to the touch
Spreadability and skin appearance
Skin feel immediately after
application
Skin feel after 5 minutes
Skin smoothness
Improvement in the general aspects
of skin
Hydration
1—very poor;
2—poor;
3—regular;
4—good;
5—excellent.
Purchase intention: ( ) Yes ( ) No
Name:

The results obtained in the sensory evaluation showed that between the two emulsions object of study formulation F5 recorded the highest grade in the parameters: Feel to the touch;

Spreadability and skin appearance;

Skin feel immediately after application;

Skin feel after 5 minutes; and

Improvement in the general aspects of skin, when compared to the formulation F5B. And compared to gel-creams formulation F7B was the one that recorded the highest grade in all parameters evaluated when compared to formulation F7.

4.1. Purchase Intention:

Regarding the purchase intention of the formulations object of study by the volunteers, it was observed that formulation F7B was the one that got greater acceptance by the volunteers, with 100% of purchase intentions, followed by formulation F5 with 90% of purchase intentions. But formulation F5B was the one that had lower acceptance by the volunteers, with 30% of purchase intentions. Thus, formulations F5 (emulsion) and F7B (gel-cream) were the formulations chosen for the evaluation tests of effectiveness.

### Example 5

### Evaluation of Short-Term Effectiveness (Immediate Effects) of the Formulations Developed, with or without Spirulina

The effectiveness of the formulations was performed with formulations F5 and F7B with or without Spirulina. We selected 14 female volunteers. For the selection of the volunteers we considered the following exclusion criteria: pregnancy or lactation; individuals with a previous history of adverse reactions to the use of cosmetics; individuals on medication likely to produce abnormal skin response; localized or generalized skin diseases; and excess hair in the areas of study. The region chosen for the studies of immediate effects of the formulations object of the study was the anterior middle part of the forearms. The volunteers were tested for effectiveness, which started 20 minutes after acclimatization in an environment with controlled temperature and air humidity, from 20 to 22° C. and from 45 to 55%, respectively. The right forearm of the volunteers was divided into two regions of approximately 36 cm², where formulations F5 (emulsion) and F5A (emulsion plus active Spirulina) were applied. The left forearm was also divided into two regions of approximately 36 cm², where formulations F7B (gel-cream) and F7B+A (gel-cream plus active Spirulina) were applied. These regions and formulations applied were randomized among the volunteers to minimize the differences between the analyzes.

In the tests of the effectiveness evaluation (short-term), we assessed the water content of the stratum corneum, the loss of trans-epidermal water, the viscoelastic properties of the skin and the cutaneous microrelief using the equipment Corneometer® CM 825, Tewameter® TM, Cutometer® SEM 575 and Visioscan® VC 98, respectively. The results are cataloged in FIG. 5.

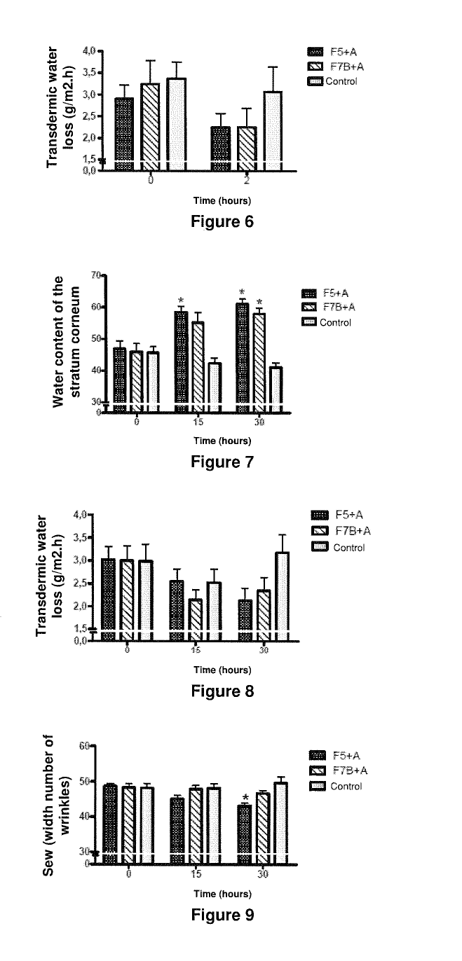

All formulations object of study significantly increased the water content of the stratum corneum, indicating an increase in skin moisturizing in the regions of the forearms. With regard to loss of trans-epidermal water, the formulations studied showed reduction effects when compared to baseline values, suggesting that the formulations object of the study showed an effect on improvement of barrier function of the skin. However, this decrease was not statistically significant because of the interindividual variation in the group. Formulation F7B+A (gel-cream plus active Spirulina) showed the most pronounced effect in reducing trans-epidermal water. FIG. 6 illustrates the evaluation of this characteristic.

In the evaluation of the viscoelastic properties of the skin, formulations F7B (gel-cream) and F7B+A (gel-cream plus active Spirulina) caused an increase in parameter values Uv/Eu, which is related to the viscoelasticity of the skin 2 hours after application of the formulations compared to baseline values. But this increase did not show any statistically significant difference due to the variability among subjects in the study group. Regarding the cutaneous microrelief, formulations caused an improvement in the skin texture, with little reduction in wrinkles and skin roughness 2 hours after the application compared to the baseline values. This reduction did not show any statistically difference due to the variability among the volunteers or due to the time of use of the formulations.

### Example 6

### Evaluation of the Long-Term Efficacy of the Formulations Containing Spirulina

The effectiveness evaluation of the formulations was performed with formulations F5 and F7B with Spirulina, F5+A and F7B+A, respectively. We selected 14 female volunteers aged between 30 and 50 years. For the selection of these volunteers were considered the same exclusion criteria that for the short-term studies. The region chosen for the studies of long-term effects of the formulations object of the study was the same anterior middle part of the forearms. The volunteers were tested for efficacy (completion of the baseline measures—T0), which started 20 minutes after acclimatization in an environment with controlled temperature and air humidity, from 20 to 22° C. and from 45 to 55%, respectively. After the tests the volunteers were instructed on the correct way and the area of application of the formulations. Each volunteer received 2 formulations F5+A and F7B+A for application twice a day in the specified regions, one region of the forearm reserved for carrying out the control measures, that is, without application of the formulation. These regions and formulations applied were randomized among the volunteers in order to minimize the differences between the analyzes. After 14 and 28 days from the date of application of the formulations, the volunteers returned to the clinical studies laboratory to carry out the measures in order to assess the effects of the formulations on the skin (T14 and T28).

In the tests of the long-term effectiveness evaluation, we assessed the water content of the stratum corneum (FIG. 7), the loss of trans-epidermal water (FIG. 8), the viscoelastic properties of the skin and the cutaneous microrelief (Sew parameter) using the equipment Corneometer® CM 825, Tewameter® TM, Cutometer® SEM 575 and Visioscan® VC 98, respectively (FIG. 9).

The formulations tested increased significantly the water content of the stratum corneum at the times of 28 days, which indicates an increase in skin hydration in the regions applied. Only formulation F5+A showed a significant increase in skin hydration at the time of 14 days. The two formulations showed a reduction in the loss of trans-epidermal water in the times of 14 and 28 days, indicating that these formulations acted improving the skin barrier function. Regarding the assessment of skin microrelief, formulation F5+A (emulsion) plus Spirulina showed a significant reduction in the number of wrinkles after 28 days of application compared to the baseline values. Furthermore, this same formulation F5+A showed a small decrease (not significant) of skin roughness. These results demonstrate that the emulsion plus Spirulina had increased beneficial effect on the skin microrelief with a reduced number of wrinkles and a tendency to reduce the roughness of the skin.

The above results pointed to a cosmetic composition containing the active of natural origin, namely, the dry extract of Spirulina produced industrially, stable and offering different benefits to the skin, with proper safety and efficacy.

1. A cosmetic composition containing Spirulina as a dry extract wherein Spirulina is present in a concentration ranging from 0.1 to 5.0% by weight, and cosmetically acceptable vehicles.

2. A cosmetic composition containing Spirulina according to claim 1, wherein the cosmetically acceptable excipients are selected from the group consisting of autoemulsifying nonionic wax, acrylate polymer, propylene glycol, glycerin, phenoxyethanol and parabens, silicone microemulsion, silicon, volatile silicon, octyl octanoate and hydrogenated and ethoxylated castor oil.

3. A cosmetic composition containing Spirulina according to claim 2, wherein the composition comprises one or more of said cosmetically acceptable excipients in the following ratios by weight: autoemulsifying nonionic wax (0 to 12.0%), acrylate polymer (0-2.0%), propylene glycol (4.0-6.0%), Glycerin (6.0%), Phenoxyethanol and parabens (0.8%), silicone microemulsion (4.0-5.0%), DC 9040 (10.0 to 21.0%), DC 245 (5.0-7.0%), DC 9011 (0-5.0%) DC 200/50 (0 to 3.0%), octyl octanoate (3.0%) and hydrogenated and ethoxylated castor oil (0 to 3.0%).

4. A cosmetic composition containing Spirulina according to claim 1, wherein the composition is formulated as an emulsion.

5. A cosmetic composition containing Spirulina according to claim 4, wherein the composition comprises, in ratios by weight, from 0.1 to 5.0% of Spirulina as a dry extract, 12% of autoemulsifying nonionic wax, 4.0% of Propylene Glycol, 6.0% of Glycerin, 0.8% of phenoxyethanol and parabens, 5.0% of silicone microemulsion, 10.0% of DC 9040, 5.0% of DC 245, 5.0% of DC 9011, 3.0% of DC 200/50, 3.0% of octyl octanoate and distilled and deionized water (qs 100).

6. A cosmetic composition containing Spirulina according to claim 1, wherein the composition is formulated as a gel-cream.

7. A cosmetic composition containing Spirulina according to claim 1, wherein the composition comprises, in ratios by weight, from 0.1 to 5.0% of Spirulina as a dry extract, 2.0% of acrylate polymer, 6.0% of Propylene Glycol, 6.0% of Glycerin, 0.8% of phenoxyethanol and parabens, 4.0% of silicone microemulsion, 21.0% of DC 9040, 7.0% of DC 245, 3.0% of octyl octanoate, 3.0% of hydrogenated and ethoxylated castor oil and distilled and deionized water (qs 100).

8. A method of cosmetic treatment, comprising applying a cosmetic composition to the skin wherein the cosmetic composition comprises Spirulina in a concentration ranging from 0.1 to 5.0% by weight, and cosmetically acceptable vehicles.

9. The method of claim 8, wherein the cosmetically acceptable excipients are selected from the group consisting of autoemulsifying nonionic wax, acrylate polymer, propylene glycol, glycerin, phenoxyethanol and parabens, silicone microemulsion, silicon, volatile silicon, octyl octanoate and hydrogenated and ethoxylated castor oil.

10. The method of claim 9, wherein the cosmetic composition comprises one or more cosmetically acceptable excipients in the following ratios by weight: autoemulsifying nonionic wax (0 to 12.0%), acrylate polymer (0-2.0%), propylene glycol (4.0-6.0%), Glycerin (6.0%), Phenoxyethanol and parabens (0.8%), silicone microemulsion (4.0-5.0%), DC 9040 (10.0 to 21.0%), DC 245 (5.0-7.0%), DC 9011 (0-5.0%) DC 200/50 (0 to 3.0%), octyl octanoate (3.0%) and hydrogenated and ethoxylated castor oil (0 to 3.0%).

11. The method of claim 8, wherein the cosmetic composition is formulated as an emulsion.

12. The method of claim 11, wherein the composition comprises, in ratios by weight, from 0.1 to 5.0% of Spirulina as a dry extract, 12% of autoemulsifying nonionic wax, 4.0% of Propylene Glycol, 6.0% of Glycerin, 0.8% of phenoxyethanol and parabens, 5.0% of silicone microemulsion, 10.0% of DC 9040, 5.0% of DC 245, 5.0% of DC 9011, 3.0% of DC 200/50, 3.0% of octyl octanoate and distilled and deionized water (qs 100).

13. The method fo claim 8, wherein the composition comprises, in ratios by weight, from 0.1 to 5.0% of Spirulina as a dry extract, 12% of autoemulsifying nonionic wax, 4.0% of Propylene Glycol, 6.0% of Glycerin, 0.8% of phenoxyethanol and parabens, 5.0% of silicone microemulsion, 10.0% of DC 9040, 5.0% of DC 245, 5.0% of DC 9011, 3.0% of DC 200/50, 3.0% of octyl octanoate and distilled and deionized water (qs 100).

14. The method of claim 8, wherein the cosmetic composition is formulated as a gel-cream.

15. The method of claim 8, wherein the composition comprises, in ratios by weight, from 0.1 to 5.0% of Spirulina as a dry extract, 2.0% of acrylate polymer, 6.0% of Propylene Glycol, 6.0% of Glycerin, 0.8% of phenoxyethanol and parabens, 4.0% of silicone microemulsion, 21.0% of DC 9040, 7.0% of DC 245, 3.0% of octyl octanoate, 3.0% of hydrogenated and ethoxylated castor oil and distilled and deionized water (qs 100).

16. A cosmetic composition containing Spirulina according to claim 1, wherein the composition comprises, in ratios by weight, from 0.1 to 5.0% of Spirulina as a dry extract, 12% of autoemulsifying nonionic wax, 4.0% of Propylene Glycol, 6.0% of Glycerin, 0.8% of phenoxyethanol and parabens, 5.0% of silicone microemulsion, 10.0% of DC 9040, 5.0% of DC 245, 5.0% of DC 9011, 3.0% of DC 200/50, 3.0% of octyl octanoate and distilled and deionized water (qs 100).
